(19) United States
(12) Patent Application Publication
(10) Pub. No.: US 2007/0104673 A1
(43) Pub. Date: May 10, 2007

(54) Base composition for cosmetic products

(76) Inventors: Maria Teresa Belmar, Shambrook (GB); Shiping Zhu, Shambrook (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/581,292

(22) PCT Filed: Nov. 19, 2004
(86) PCT No.: PCT/EP04/13225

§ 371 Date: Jun. 1, 2006

(30) Foreign Application Priority Data
Dec. 4, 2003 (EP) 03257648.0

(51) Int. Cl. A61K 8/37

(52) U.S. Cl. 424/70.31

(57) ABSTRACT

A cosmetic composition comprising (i) less than 10 wt % water, (ii) fatty acid, (iii) a structuring agent selected from the group comprising glycerine monostearate and glycol monostearate or a combination thereof, and (iv) at least 2 wt % neutralised fatty acid is a suitable base composition for forming a cosmetic product on hydration, such as a cream or lotion.

The invention relates to a base composition for cosmetic products that easily hydrates to form a smooth product.

### BACKGROUND AND PRIOR ART

Anhydrous products that can be hydrated to form a cosmetic composition such as a skin care composition or lotion are known in the art.

U.S. Pat. No. 6,221,364 discloses a composition which is essentially free of water which is suitable to be mixed with water with or without additives for forming a cream or a lotion. Advantages of such compositions are disclosed to be avoiding degradation of active ingredients, stability for storage, resistance towards growing rancid of the oil components.

U.S. Pat. No. 5,607,666 discloses powders that have been obtained from a homogenised and dehydrated oil in water emulsion containing a) a structuring and emulsifying agent, B) at least one fat, c) at least one cosmetically active substance, and d) an aqueous phase.

The products disclosed in these patents are suitable for rehydration but there is a need for improved compositions with respect to the speed of hydration, ease of preparation of the hydratable composition and rheology and homogeneity of the reconstituted product. Often reconstituted products are too fluid to be used as a cream and/or show lumps of particles.

WO02003/097003 relates to compositions suitable for rehydration. Those compositions rely on the combination of fatty acid and an organic base wherein the fatty acid is at least partially neutralised. Although this disclosure mentions that some inorganic base may be present, there is no disclosure of its combination with glycerine or glycol monostearate.

Although this document mentions that inorganic basis are unsuitable for providing a composition that may be rehydrated in cold water, we have now found circumstances under which an inorganic base can be used to provide rehydratable compositions that result in a smooth cosmetic composition with consistency from cream to lotion, when mixed with cold water.

Accordingly, the invention provides a cosmetic composition comprising (i) less than 10 wt % water, (ii) a structuring agent selected from the group comprising glycerine monostearate and glycol monostearate or a combination thereof, and (iii) at least 2 wt % neutralised fatty acid.

In a further aspect, the invention provides a method for the preparation of such a composition, wherein an inorganic base, a fatty acid and a structuring agent selected from the group comprising glycerine monostearate and glycol monostearate or a combination thereof are mixed, heated to a temperature from 80 to 120° C., cooled to a temperature below 40° C. and formed into a powder, tablet or amorphous mass.

In a further aspect, the invention provides a process of hydrating such a composition to form a skin cream, shampoo or lotion, wherein the composition is mixed with water at a temperature of from 0 to 35° C. at a weight ratio of from 1:3 to 1:20.

### DETAILED DESCRIPTION OF THE INVENTION

Unless otherwise indicated all percentages are by weight.

The Cosmetic Composition

The invention relates to an essentially water free composition which may be hydrated to form an end-user cosmetic composition. The composition according to the invention is preferably in the form of a tablet, powder or amorphous mass, most preferred in the form of a powder. Thus, the composition comprises less than 10 wt % water, preferably less than 5 wt %, more preferred less than 1 wt % water.

Structuring Agent

The composition comprises a structuring agent. Structuring agents are common ingredients for cosmetic products and are added to contribute to shear thinning properties to aid in spreading of the product, to sensory perception on skin surface and to contribute to a yield value to aid suspension of other ingredients.

The structuring agent provides structure to the final product after re-hydration of the composition. The structuring agent is selected such that it swells in the presence of cold water and thus imparts firmness to the rehydrated product. The composition and type of structuring agents are preferably selected such that after hydration a product results which is characterised by an apparent viscosity of from 0.1 to 10000 Pa·s at a shear rate of 10 s⁻¹ to 100 s⁻¹ or a yield stress of from 0.1 to 10.000 Pa, preferably from 1 to 10.000 Pa, more preferred from 10 to 1.000 Pa.

The structuring agent is selected from the group comprising glycerine monostearate and glycol monostearate or a combination thereof. The most preferred structuring agent is glycerine monostearate.

The amount of structuring agent may vary, depending on the type of final composition that is aimed at. In a preferred embodiment, the amount of structuring agent is from 20 to 90 wt % on the weight of the essentially water free composition.

More suitable amounts are from 15 to 70 wt %, more preferred from 20 to 60 wt %.

It is important that the structuring agent swells in the presence of cold water.

Fatty Acid

The composition also comprises fatty acid. The fatty acid serves to provide anionic charges after being neutralized by the base and co-crystallize with the structuring agent. These mixed crystals were found to swell in cold water.

The fatty acid may be added as such or may be part of one of the ingredients e.g. as a by-product or impurity.

In certain preferred embodiments, the hydrocarbon chain length of the fatty acid used is from 14 to 22, preferably 14 to 20, more preferably 16 to 18 carbon atoms.

Preferred fatty acids are selected from the group comprising lauric acid, myristic acid, palmitic acid, stearic acid, oleic acid and combinations thereof.

The swelling properties may furthermore be influenced by the relative amount of the fatty acid and the structuring agent. Therefore it is preferred that the amount of fatty acid is from 5 to 90 wt % based on the total amount of fatty acid and structuring agent.

Neutralised Fatty Acid

It was found that the amount of neutralised fatty acid determines the level of swelling. At a too low level of neutralised fatty acid, the swelling is reduced and the rehydrated products easily form lumps or other inhomogeneous parts. If the amount of neutralised fatty acid is too high then the rehydrated product possesses a pH above 10.

In a preferred embodiment the amount of neutralised fatty acid is from 2 to 15 wt %, more preferred from 2.5 to 8 wt %.

The pH of the composition when suitably hydrated is preferably below 9, which is desired for skin cream products. The pH obtained may be different, depending on the amount of neutralised fatty acid and the ratio between neutralised and non-neutralised fatty acid. Therefore, optionally the product comprises an acidulant or base to obtain the desired pH below 9 after rehydration.

Preferably products that merely comprise neutralised fatty acid comprise a base to lower the pH of a rehydrated sample to below 9. Preferably the amount of acid/base is such that the pH of a rehydrated composition is between 6 and 9.

Other Optional Ingredients

The composition optionally comprises other ingredients which are generally known for inclusion in cosmetic formulations. Preferred compositions therefore comprise an ingredient selected from the group comprising emulsifying agent, surfactant, other structuring agent than glycerine monostearate and glycol monostearate, emollient oils and waxes, humectants, functional ingredients, preservatives, antioxidants, chelating agents, perfume, colouring agent or a combination thereof.

Emulsifying agents or surfactants may be classified as:
a) anionic surfactants such as carboxylates, alkylsulfate, ethoxylated alkyl sulfates, sulfosuccinates, isethionates, taurates, phosphated esters and lactylates;
b) cationic surfactants such as fatty quaternary ammonium salts, pyridinium compounds, ethoxylated amines, imidazolines, amidoamines and phospholipids (
c) nonionic surfactants such as fatty alcohols (e.g. cetylalcohol), fatty alcoholesters, polyethylene glycol carboxylates, sterols, sterol ethoxylates, sugar ethoxylates, polyethylene glycol and polypropylene glycol polymers;
d) amphoteric surfactants. These surfactants are less preferred, but a small amount may be present.

Any of these or a combination thereof may be present in the claimed composition. The most preferred compositions comprise cetylalcohol.

Examples of other structuring agents are waxes, gelled oils, thickening agents such as gums, polysaccharides, pectin, synthetic polymers e.g. Carbopol, clays.

Emollient oils and waxes may be added to impart a specific function to the end-product composition such as occlusion, sensory properties e.g. skin feel especially after-feel. Examples of emollient oils and waxes are mineral oils, isopropyl palmitate, isopropyl myristate, propylene glycol diisostearate.

To reduce the symptoms of dry skin, the compositions preferably comprise a humectant. Suitable humectants are eg selected from the group comprising polyols such as glycerol, propylene glycol, sorbitol, diglycerol, isoprene glycol; natural moisturizing agents such s lactate, urea, pyrollidone carboxylate and aminoacids.

Various types of additional active ingredients may be present in compositions of the present invention. Actives are defined as skin benefit agents other than emollients and other than ingredients that merely improve the physical characteristics of the composition. Although not limited to this category, general examples include additional anti-sebum ingredients such as talcs and silicas, and sunscreens. Further examples include silk protein, fragrances, colouring agents, healthy skin ingredients such as AHA, collagen, amino acids; vitamins such as vitamin A and vitamin E, triple lipids such as lecithin, soy sterol; or combinations thereof.

Process for Making the Composition

The composition can conveniently be made by blending an inorganic base with a fatty acid to produce neutralised fatty acid, leaving some fatty acid unneutralised. Suitable inorganic bases are e.g. sodium hydroxide and potassium hydroxide. Most preferred the inorganic base is potassium hydroxide. In a preferred embodiment, the amount of inorganic base is from 0.1 to 4 wt %, preferably from 0.5 to 3 wt % on total weight of the essentially water free composition.

In this embodiment, the degree of neutralisation of the fatty acid is preferably at least 50% at a total fatty acid concentration of from 5 to 10 wt %. The degree of neutralisation is preferably from 5 to 40% at a total fatty acid concentration of from 20 to 80 wt %.

Conveniently, the essentially water free composition is a simple mechanical mixture, optionally heated, and may conveniently be in the form of a tablet, powder or amorphous mass.

It is preferred that the structuring agent and the neutralised fatty acid are co-crystallised in the essentially water free composition.

Therefore the essentially water free composition is preferably prepared in a process wherein the structuring agent and the neutralised fatty acid are together submitted to a temperature treatment of at least 80° C. Without wishing to be bound by any theory, it is believed that this heat treatment induces the formation of an isotropic solution which results in co-crystallizing of the structuring agents and the neutralised fatty acid on cooling.

The mixing with aqueous base may take place in any manner but is preferably manual by stirring or shaking.

The essentially water free composition according to the invention may be prepared in any suitable way. It is advantageous that there is generally no need for excessive heating or complicated process steps to make the composition. In a preferred method, the constituents are mixed, heated to a temperature from 20 to 80° C., cooled to a temperature below 40° C., preferably below 20° C. and preferably formed into a powder or tablet.

Hydration

The composition of the present invention can be hydrated to provide an aqueous cosmetic base or final product. These products preferably are spreadable and/or pourable and most preferred have a rheology as exemplified by the above indicated apparent viscosity values.

One of the advantages of the method according to the invention is that it enables the use of cold water with a temperature from 0 to 35° C. for the preparation of the end-user composition. A cosmetic composition generally comprises from 60 to 90 wt % water and hence this provides a saving in energy cost as the large water volumes need not be heated before use. Also if the method is carried out on small scale by a consumer, the consumer need not use hot water and electricity for the preparation which may for example not always be available in low income countries. It is however to be noted that the aqueous base can also have a temperature above 35° C. and hence the method according to the invention provides a variety of options.

The final products are preferably prepared by a method wherein the essentially water free composition is mixed with an aqueous base at a temperature of below 80° C., more preferred from 80 to 20° C.

In the context of the invention an aqueous base is an aqueous medium which mainly comprises water but may also comprise other ingredients. The preferred aqueous base is water. Other examples include tea, juices, water with supplemental ingredients such as colouring agent, ingredients with a health benefit, flavour ingredients.

The cosmetic products suitable for the end-user (consumer) typically comprise 60-100%, preferably 60 to 99 wt % of aqueous cosmetic base (eg. water, glycerin monostearate and partially neutralized fatty acid), the balance comprising other components necessary to provide the desired form of product e.g. a topical skin care composition.

To enable customisation of the resulting cosmetic composition, part of the optional ingredients are preferably added together with aqueous base or after the aqueous base has been added.

This applies specifically for benefit agents and unstable ingredients.

End Use

The aqueous cosmetic base or essentially anhydrous composition can be provided to a consumer for individual preparation of a cream in any suitable way. Preferably a kit of parts is provided for preparing a cosmetic product, said kit comprising a cosmetic container, an essentially anhydrous composition according to the invention and instructions for use of the kit. Said instructions will include guidance on the temperature of the aqueous base and amount of aqueous base to be added for one unit of anhydrous composition, optional further ingredients that may be added and the desired mode of agitation of the mixture.

Preferably the cosmetic container is provided with measuring signs to show to the consumer the amount of water that is best added to prepare a final cosmetic product.

To accommodate the desire for customisation, preferably the kit of parts additionally comprises a separate packaging unit comprising a skin cream additive, preferably selected from the group comprising perfume, retinol, colorant, oils, herbs, vitamins or a combination thereof. Alternatively such additive may also be part of the essentially anhydrous composition. According to another embodiment said skin cream additive is mixed with an aqueous base which is part of the kit of parts. Said aqueous base optionally comprises (part of) the additives.

The invention is now illustrated by the following non limiting examples.

### EXAMPLES

### Example 1

TABLE 1
Anhydrous base composition

| | Wt % (before | Wt % (after |
|---|---|---|
| Ingredients | mixing) | mixing) |
| Glycerin monostearate | 58.7 | 58.7 |
| Cetyl alcohol | 5 | 5 |
| Fatty acid | 25 | 18.7 |
| Glycol monostearate | 8.7 | 8.7 |
| KOH* | 1.3 | 0 |
| Water | 1.3 | 1.7 |
| Potassium soap | 0 | 7.2 |
| Total | 100 | 100 |

*In order to facilitate the neutralization reaction at 80° C. KOH is added as a 50% aqueous solution.

All ingredients of table I were mixed and melted at 80° C. Subsequently the mixture was cooled to about 20° C. and then made into a powder.

The degree of neutralisation was 25% so the total level of neutralised fatty acid was about 7.2 wt %. 90% of water was mixed with 10% of powder using a spatula at room temperature.

A smooth, homogeneous skin cream base resulted.

### Example 2

TABLE 2

| | Wt % (before | Wt % (after |
|---|---|---|
| Ingredients | mixing) | mixing) |
| Glycerin monostearate | 14.0 | 14.0 |
| Cetyl alcohol | 3.3 | 3.3 |
| Fatty acid | 21.0 | 14.3 |
| Glycol monostearate | 5.8 | 5.8 |
| KOH* | 1.4 | 0 |
| Water | 1.4 | 1.8 |
| Glycerin | 24.8 | 24.8 |
| Mineral oil | 23.3 | 23.3 |
| Fragrance | 1.7 | 1.7 |
| Titanium dioxide | 0.8 | 0.8 |
| Dimethicone | 2.5 | 2.5 |
| Potassium soap | 0 | 7.7 |
| Total | 100 | 100 |

*In order to facilitate the neutralization reaction at 80° C. KOH is added as a 50% aqueous solution

All ingredients of table I were mixed and melted at 80° C. Subsequently the mixture was cooled to about 20° C. and then made into a powder.

The degree of neutralisation was 33% so the total level of neutralised fatty acid was 7.7 wt %.

A full formulation cream can be prepared by mixing 18% of the composition with 82% water.

### Example 3

TABLE 3

| | Wt % (before | Wt % (after |
|---|---|---|
| Ingredients | mixing) | mixing) |
| Glycerin monostearate | 47.6 | 47.6 |
| Cetyl alcohol | 7.1 | 7.1 |
| Fatty acid | 31.0 | 19.1 |
| Glycol monostearate | 9.5 | 9.5 |
| KOH* | 2.4 | 0 |
| Water | 2.4 | 3.1 |
| Potassium soap | 0 | 13.6 |
| Total | 100 | 100 |

*In order to facilitate the neutralization reaction at 80° C. KOH is added as a 50% aqueous solution.

All ingredients of table 3 were mixed and melted at 80° C. Subsequently the mixture was cooled to about 20° C. and then made into a powder. The degree of neutralization was 40% so the total level of neutralised fatty acid was 13.6 wt %.

A full formulation cream was prepared by mixing 9% of the composition with 86% water, 3% glycerin, 2% sunflower seed oil.

1. A cosmetic composition comprising:
(i) less than 10 wt % water
(ii) fatty acid,
(iii) a structuring agent selected from the group comprising glycerine monostearate and glycol monostearate or a combination thereof, and
(iv) at least 2 wt % fatty acid neutralized by an inorganic base.

2. Cosmetic composition according to claim 1, wherein the amount of neutralised fatty acid is from 2 to 15 wt %.

3. Cosmetic composition according to claim 1, wherein the structuring agent is glycerine monostearate.

4. Cosmetic composition according to claim 1, wherein the amount of fatty acid is from 5 to 90 wt % based on the total amount of fatty acid and structuring agent.

5. Cosmetic composition according to claim 1, wherein the hydrocarbon chain length of the fatty acid is from 14 to 20, preferably from 16 to 18 carbon atoms.

6. Cosmetic composition according to claim 1, wherein the amount of structuring agent is from 15 to 75 wt % on total weight of the composition.

7. Cosmetic composition according to claim 1, wherein the total amount of fatty acid is from 5 to 80 wt % on total weight of the composition.

8. Cosmetic composition according to claim 1, which further comprises an ingredient selected from the group comprising emulsifying agent, other structuring agent than glycerine monostearate and glycol monostearate, emollient oils and waxes, humectants, functional ingredients, preservatives, antioxidants, chelating agents, perfume, colouring agent or a combination thereof.

9. Method for the preparation of a composition according to claim 1, wherein an inorganic base, a fatty acid and a structuring agent selected from the group comprising glycerine monostearate and glycol monostearate or a combination thereof are mixed, heated to a temperature from 80 to 120° C., cooled to a temperature below 40° C. and formed into a powder, tablet or amorphous mass.

10. A process of hydrating a composition according to claim 1 to form a skin cream, shampoo or lotion, wherein the composition is mixed with an aqueous base at a temperature of below 80° C.

11. A process according to claim 10, wherein the temperature of the aqueous base is from 0 to 35° C.

12. A process according to claim 10, wherein the composition and aqueous base are mixed together in a weight ratio of from 1:3 to 1:20.
